(19) United States
(12) Patent Application Publication
(10) Pub. No.: US 2022/0273468 A1
(43) Pub. Date: Sep. 1, 2022

(54) Anatomically Aligned Prosthetic Ankle

(71) Applicant: LOMA LINDA UNIVERSITY, Loma Linda, CA (US)

(72) Inventors: Michael Davidson, Loma Linda, CA (US); Spencer Cutting, Loma Linda, CA (US)

(73) Assignee: LOMA LINDA UNIVERSITY, Loma Linda, CA (US)

(21) Appl. No.: 17/753,007

(22) PCT Filed: Aug. 11, 2020
(86) PCT No.: PCT/US2020/045697

§ 371 Date: Feb. 15, 2022

Related U.S. Application Data
(60) Provisional application No. 62/888,587, filed on Aug. 19, 2019.

(51) Int. Cl. A61F 2/66; A61F 2/74

(52) U.S. Cl. CPC A61F 2/6607; A61F 2/74; A61F 2002/6614; A61F 2002/5038

(57) ABSTRACT

The present disclosure relates to an anatomically aligned prosthetic ankle with a passive assist and associated methods. The prosthetic ankle includes a talus movably coupled to a tibia section by a connector about which the talus is pivotal such so during locomotion by a user of the prosthetic ankle. The passive to assist provides a selected dorsi-flexion force to aid in the push-off or pre-swing phases of gait for the user.

### CROSS REFERENCE TO RELATED APPLICATION

This application is a PCT and claims priority to and the benefit of U.S. Provisional Application No. 62/888,587, filed Aug. 19, 2019, titled “ANATOMICALLY ALIGNED PROSTHETIC ANKLE”, the entire disclosure of which is incorporated herein by reference.

### FIELD OF THE DISCLOSURE

The present disclosure generally relates to prosthetic replacement limbs and associated methods. In particular, the present disclosure relates to a prosthetic ankle with a passive pneumatic assist, and which is designed to substantially anatomically align with a user's natural or existing ankle physiology and improve push-off and/or a pre-swing phase of the user's gait.

### BACKGROUND

It is estimated that there are upwards of two million persons living in the United States alone who suffer from limb loss. Prosthetic devices have been used for quite some time as a replacement for amputated limbs to try to help those suffering from limb loss recover at least some degree of movement lost by such amputated limbs. While improvements in prosthetic devices have been made over the years, the problem with many prosthetic devices has been an inability to substantially closely replicate a person's natural or normal (pre-loss) movement. For example, the human ankle serves as an important component for walking, navigating slopes, and even simpler tasks such as sitting, squatting and standing. In particular, ankle movement is important for providing the push-off or pre-swing phase of a person's gait, i.e., the initial force exerted when a person stands or starts to walk, as well as their balance during locomotion, particularly when moving over uneven ground or up a slope, ramp or stairs. However, even with more advanced and/or sophisticated prosthetic legs, the loss of an ankle to amputation continues to present difficulties in walking and locomotion, impeding balance, ramp and stair safety, and increasing the potential for falling. Often, patients must learn to walk in a different manner and, in effect, retrain their brains to initially recognize their limb/prosthetic foot is missing and focus on walking in a different manner, taking into account the prosthesis, particularly when first beginning with their new leg prosthesis.

Accordingly, it may be seen that a need exists for a prosthetic ankle that is anatomically aligned and/or matched with the user's natural or existing ankle physiology, and which addresses the foregoing and other related and unrelated problems in the art.

### SUMMARY

Briefly described, the present disclosure generally is directed to a prosthetic ankle for use as a replacement ankle and/or lower leg in bilateral amputations. In one aspect, the present disclosure is directed to an anatomically aligned, prosthetic ankle with a passive dorsi-flexion tendon assist. The ankle may include a tibia section, a talus or ankle joint portion, a foot coupled to the talus, and a dorsi-flexion tendon assist mechanism that links to and extends between the talus and the tibia section. The talus and tibia section further are movably coupled or connected together by a connector such as a rod or pin, which enables rotational or pivotal movement of the talus, and the prosthetic foot which is connected thereto, with respect to the tibia section.

The connector will extend through the talus and a portion of the tibia section at an offset alignment sufficient to orient the talus at an angle or an arrangement that is shifted and/or offset from both a transverse plane and a coronal plane. Typically, the coronal alignment and transverse alignment of the talus will be selected so as to substantially match the user's natural ankle joint alignment/physiology, i.e., the prosthetic talus may be arranged with a coronal and transverse alignment that is based on or substantially matches a coronal and transverse alignment of the user's ankle or their existing, sound limb, otherwise selected so as to substantially mimic the natural transverse and coronal ankle axes of the wearer or user.

In one aspect, the talus may be arranged with a coronal alignment of approximately 5°-6° to about 10°-12° (for example, approximately 8°) from a vertical tibial midline of the user, and with a transverse alignment of approximately 5°-6° to about 10°-12°(for example, approximately 8°) external rotation with respect to the user's knee axis. Alignment of the talus further may be adjusted in both the coronal and transverse planes as needed to substantially match the ankle joint orientation of the user's sound limb for a more anatomically realistic alignment and performance, using standard adapters. In addition, the lateral ankle joint axis defined through the connector will be substantially in direct vertical alignment with the user's hip and knee joints.

In one aspect, the dorsi-flexion tendon mechanism may include a passive spring assist mechanism, such as a pneumatic or hydraulic cylinder, spring/biasing mechanism, or other similar passive assist device operable to provide a desired push-off force sufficient to assist the user in standing, initiating locomotion and other similar movement. For example, a pneumatic spring including a cylinder configured to produce a preset or adjustable force assist, and having an extensible cylinder rod or linkage, may be mounted to the tibia section, with a distal end of the cylinder rod of the spring assist mechanism generally being coupled to the talus.

Other arrangements and passive assist mechanisms also may be used. For example, the prosthetic ankle may be configured with multiple spring assist mechanisms. This may include a pair of adjacent or offset cylinders or other spring assist mechanisms mounted along the prosthetic ankle; and/or front and rear spring assist mechanisms that are configured to supply cooperative and/or adjustable or varying spring assist forces along front or rear potions of the prosthetic ankle as needed to help provide adjustable dorsi-flexion assistance and resistance and/or plantar assistance to the user in a terminal stance as well as during ambulation.

In addition, the passive assist force(s) provided by the spring assist mechanism(s) may be adjusted to vary the push-off force provided by the user's prosthetic foot during a pre-swing movement, as well as to sufficiently dampen plantarflexion movement as part of the user's standing or gait motion, based on the user's existing/sound ankle, and/or as the user adjusts to the prosthetic ankle.

The prosthetic foot is mounted along a lower portion of the talus using a connector. The foot generally will be substantially aligned with the knee and hip joints of the user and may comprise a commercially available prosthetic foot, or may be a specially designed prosthetic foot configured based on the user's physical dimensions. The prosthetic foot further may be provided with cushioning or dampening features and its orientation with respect to the talus may be adjusted as needed to more closely match the natural physiology and walking gait of the user.

Various objects, features and advantages of the present disclosure will become apparent to those skilled in the art upon a review of the following detailed description, when taken in conjunction with the accompanying drawings.

### BRIEF DESCRIPTION OF THE DRAWINGS

FIG. 1 is a perspective view of the anatomically aligned pneumatic prosthetic ankle according to the principles of the present disclosure.

FIG. 2 is a partially exploded view of the prosthetic ankle of FIG. 1.

FIG. 3 is a perspective view of the prosthetic ankle of FIGS. 1-2.

FIG. 4 is an end exploded view of the prosthetic ankle of FIGS. 1-3.

FIG. 5A is a side elevational view of the prosthetic ankle of FIGS. 1-4.

FIG. 5B is an end elevational view of the prosthetic ankle of FIGS. 1-5A.

FIGS. 6A-6B are top and bottom end views of the prosthetic ankle of FIGS. 1-5B.

FIG. 7 is a picture illustrating an embodiment of the prosthetic ankle of FIGS. 1-3.

FIG. 8 is a perspective view of an additional embodiment of a prosthetic ankle according to the principles of the present disclosure.

FIG. 9 is an exploded view of the prosthetic ankle of FIG. 8.

FIG. 10 is a side elevational view of the prosthetic ankle of FIGS. 9-10.

Those skilled in the art will appreciate and understand that, according to common practice, the various features of the drawings discussed below are not necessarily drawn to scale, and that the dimensions of various features and elements of the drawings may be expanded or reduced to more clearly illustrate the embodiments of the present disclosure as described herein.

### DETAILED DESCRIPTION

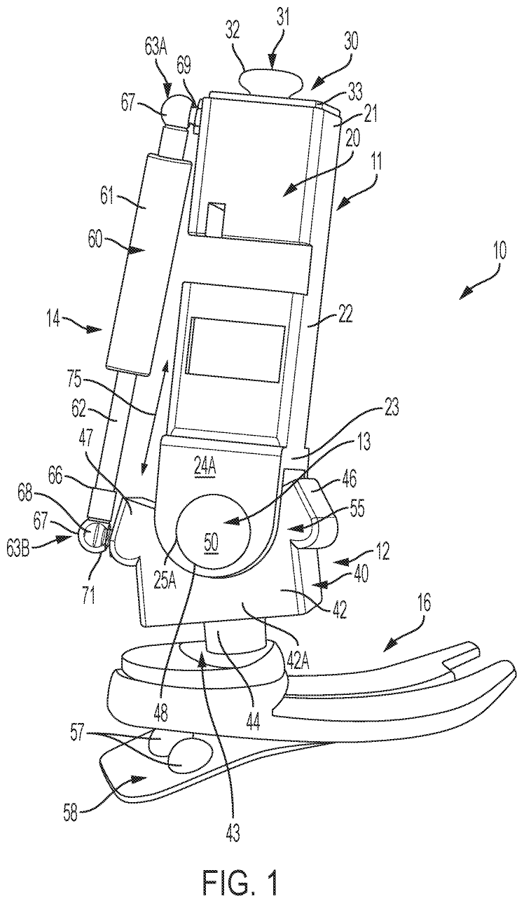

In the drawings, like numerals indicate like parts throughout the several views, and FIGS. 1-7 illustrate aspects of an anatomically aligned prosthetic ankle 10 for use as a replacement limb for patients or users that have had bilateral amputations necessitating replacement of a lower leg portion of such a patient or user. Various futures and aspects of the prosthetic ankle 10 are illustrated in the Figures and are described in further detail below. As illustrated in FIGS. 1-5B, prosthetic ankle 10 will include a tibia section 11, a talus or ankle joint 12, moveably/pivotally connect to the tibia section, a dorsi-flexion assist mechanism 14, and a prosthetic foot 16 coupled to the talus. The talus and tibia sections may be formed from durable materials such as plastics, composites, aluminum, and other, similar lightweight materials as will be understood by those skilled in the art.

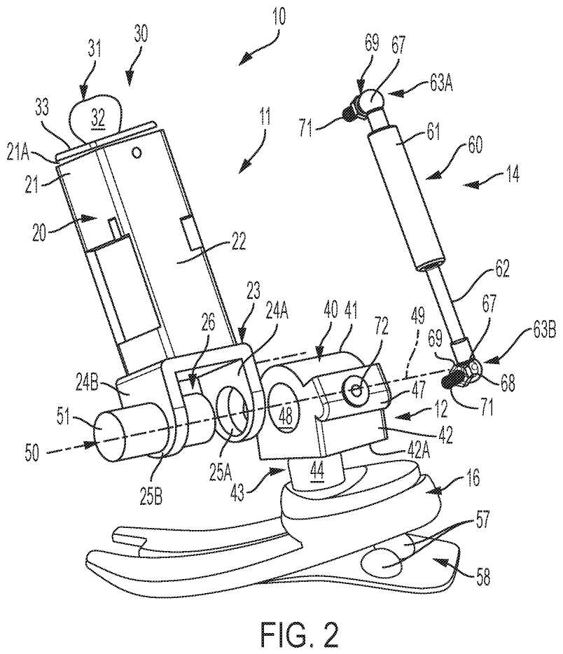

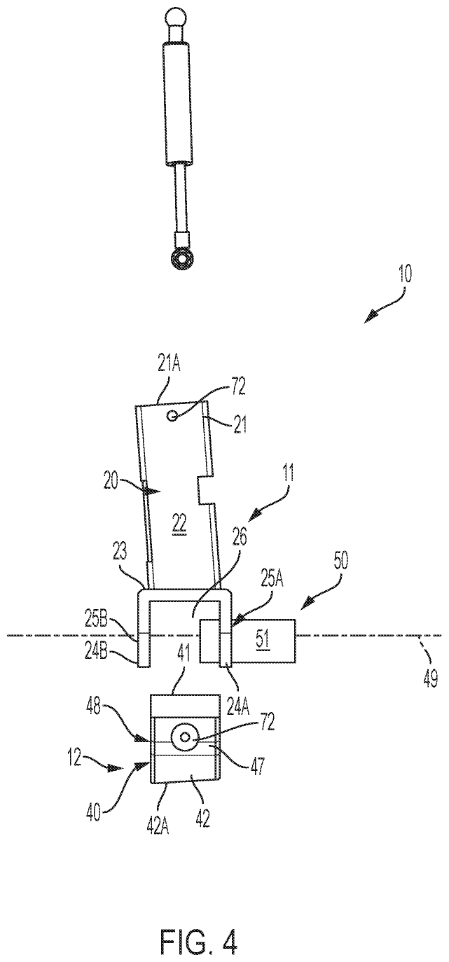

The tibia section 11, as illustrated in FIGS. 1-5B, will include an elongated body 20 that may be sized and/or otherwise configured to match the user's physiology, for example, being of a length approximately equivalent to the user's sound or existing tibia and with a configuration or structure to support user's weight. As generally illustrated in FIGS. 2 and 4, the body 20 of the tibia section 11 may have an upper or proximal end 21, an intermediate body section 22, and a lower or distal end 23. The lower or distal end 23 of the tibia section 11 further may formed as a clevis or with a substantially C or U-shaped construction, including a pair of downwardly projecting side portions or legs 24A/B. The legs 24A/B of the lower end 23 of the tibia section are spaced apart sufficient to define an open area or recess 26 within which the talus 12 of the prosthetic ankle 10 will be received, as indicated in FIGS. 1-4, and will have aligned openings 25A/B defined therethrough.

The talus generally will be received within the recess 26 defined by the legs 24A/B of the lower end 23 of the tibia section body 20 in a substantially fitted arrangement that enables rotation or pivoting movement of the talus with respect to the tibia section and in at least one direction along a substantially laterally oriented joint access, as indicated at 27. The talus 12 further may be oriented at a slight angle or cant with respect to the immediate and upper portions 22/21 of the tibia section body as indicated in FIG. 4.

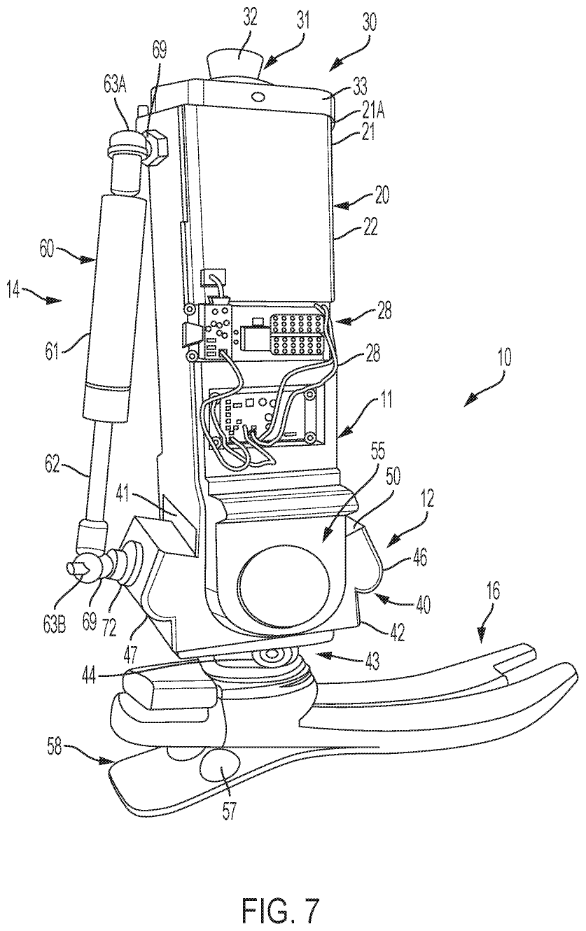

As further illustrated in FIG. 7, the body 20 of the tibia section may house or include various sensors 28, or other measurement devices that may connect, such as via a wireless/Low Energy Bluetooth connection, or by a wired connection, to a control device (not shown). The control device may connect to and receive feedback and/or positional, strain and other data from such sensors during use of the prosthetic ankle i.e. through an app, enabling tracking and recording of monitored information as to the wearer's gait and other acts of locomotion. The information developed and provided by the incorporated sensors may be used to make adjustments to the prosthetic ankle as needed.

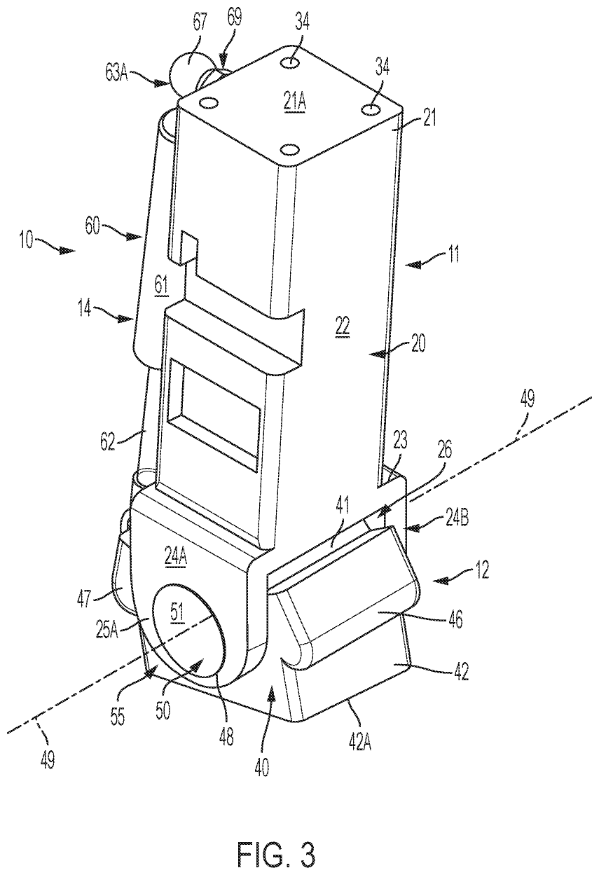

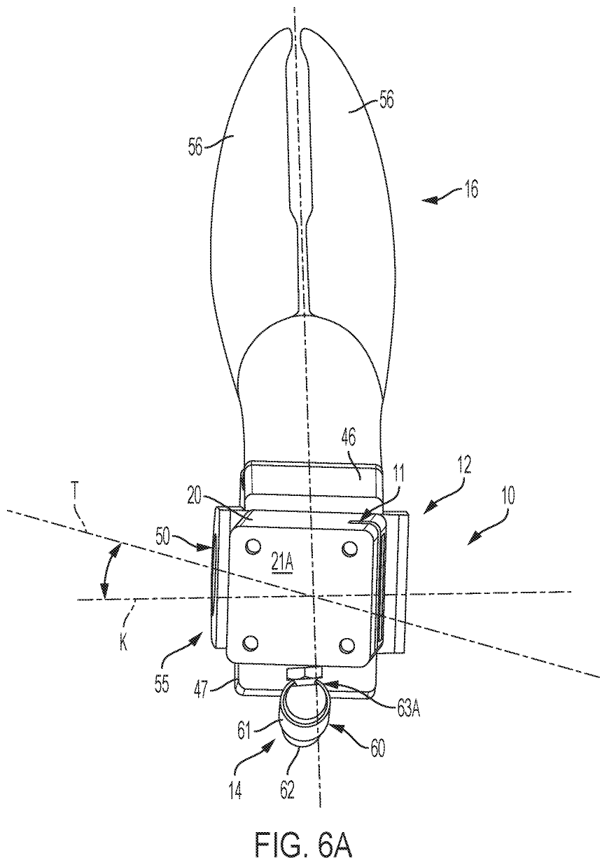

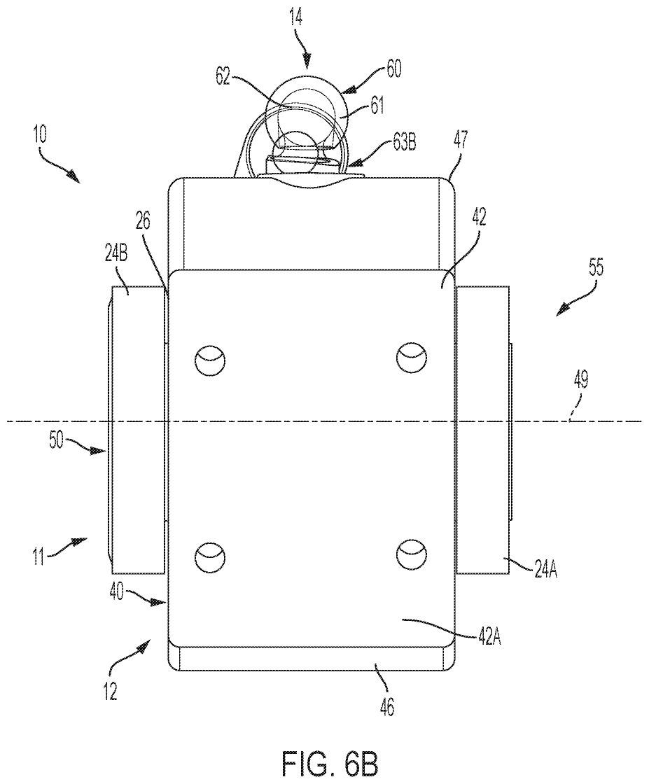

As indicated in FIGS. 1-3, 5A and 6A, the upper or proximal end 21 of the body 20 of the tibia section 11 may be figured to receive a mount or coupling device 30, such as a pyramid adapter 31, or other, similar connector or coupling device for attachment to sleeve or corresponding mating connector attached at the user's knee. In one example aspect, the connector 31 may include a body 32, mounted to a plate 33 that mounts to the upper end 21 of the body 20 of the tibia section 11, such as by fasteners. While FIGS. 3 and 6A generally show a series of fastener openings 34 at the corners of the upper surface 21A of the tibia section for connection to the coupling device 30, it will be understood by those skilled in the art that other arrangements of fasteners and other means of connecting or mounting the coupling device to the upper end of the body of the tibia section also may be used.

As further illustrated in FIGS. 1, 2, 4 and 5A-5B, the talus 12 generally will comprise a body 40 having a generally rounded or curved upper end 41, a substantially flat lower end 42. The bottom surface 42A of the lower end 42 generally will be configured to attach or mount to a corresponding coupling device 43, such as a pyramid adaptor or similar coupling device 44 for connecting the prosthetic foot 16 to the talus 12. The talus also generally will be configured so as to be compatible with a variety of different size and/or shape adapters, including a variety of commercially available adapters and/or specially designed or configured adapters. Thus, additional adjustments may be made to the coronal, transverse and lateral orientations of the talus, including adjustments in the field and/or during use by a patient, such as for mounting and substitution of different feet or for different activities. The talus further may include front and rear projecting portions 46 and 47 that may have a rounded configuration or other configuration substantially matching an anatomical talus. A central opening or passage 48 further will be defined through the body of the talus, extending substantially laterally therethrough and defining a lateral ankle joint axis indicated by line 49.

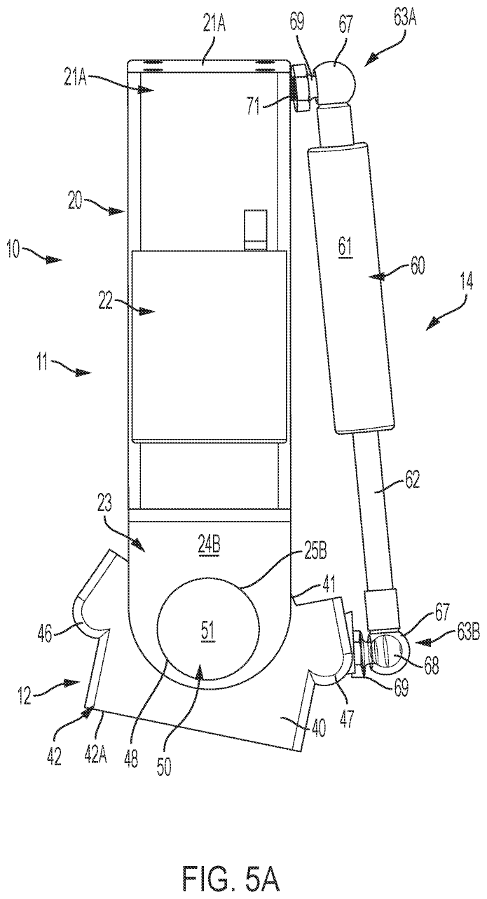

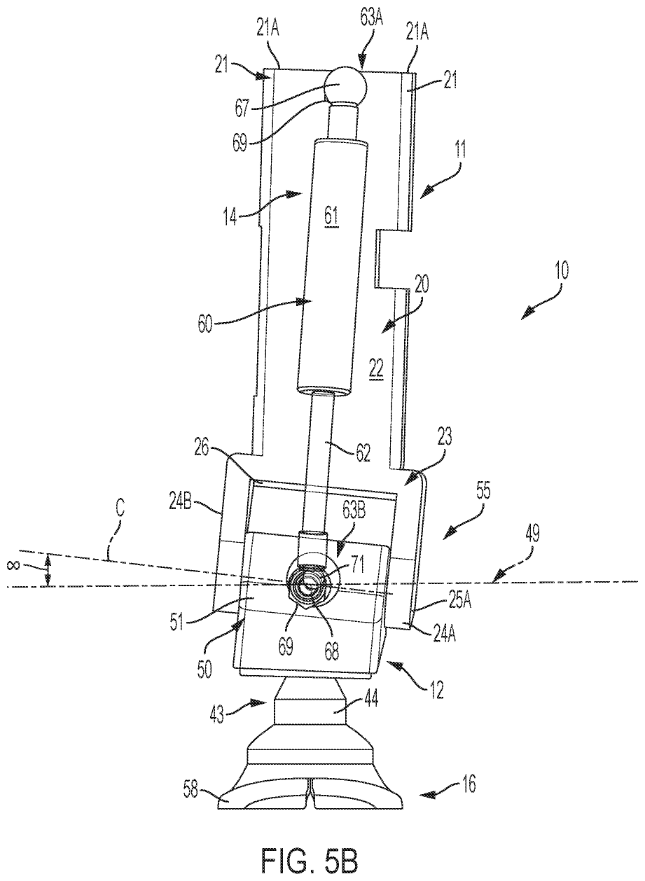

As indicated in FIGS. 2 and 4, the body 40 of the talus 12 will be received within the recess 26 defined by the downwardly projecting legs 24A/B of the body 20 of the tibia section 11, with the central opening or passage 48 defined through the body of the talus being substantially aligned with corresponding openings or passages 25A/B formed in the downwardly protruding leg portions 24A/B of the body 20 of the tibia section 11, and with the front and rear projecting portions of the talus body further projecting past front and rear portions of the tibia section body, as shown in FIGS. 1 and 5A-5B. The rounded upper portion 41 of the body 40 of the talus 12 generally will project within the recess defined by the legs of the body of the tibia section with a sufficient clearance to enable a pivoting or rotating movement of the talus about the lateral ankle joint axis 49 defined through the coincident or aligned passages or openings of the portions of the tibia section body and the passage of the talus. The forward and rearward projecting portions 46 and 47 of the talus may help provide a limit of the pivoting movement of the talus with respect to the tibia section.

A connector 50 generally will be received through the passage 48 and aligned openings 25A/B of the legs 24A/B of the body 20 of the tibia section 11 and the talus 12. The connector 50 may include a pin or rod 51 that extends along the lateral ankle joint axis 49 projecting through the tibia section and talus, as indicated in FIG. 4. The connector pin or rod thus provides a rotatable connection between the talus and tibia section about which the talus is pivotable with respect to the tibia section. As indicated in FIGS. 4, 5B and 6A, the resultant connection between the tibia section and talus defines an ankle joint 55 that additionally is off-set with respect to both a transverse plane (as indicated in FIG. 5A) and with respect to a coronal plane (as illustrated in FIG. 6A).

The ankle joint 55 will be oriented in an off-set alignment with respect to a vertical tibia midline and with respect to an axis of rotation of the user's knee, which off-set or angle of alignment generally will be selected to substantially match the user's physiology/anatomy; for example, generally matching or being based upon the user's existing or sound ankle joint. In one aspect, as illustrated in FIG. 5B, the ankle joint may be oriented to define a coronal ankle joint axis “C” having an off-set/alignment angle of approximately 8° with respect to the lateral joint axis 49, which off-set or angle may vary between plus or minus approximately 3.6 degrees (or more depending on the user's physiology). The ankle joint further may have a transverse ankle joint axis T (FIG. 6A) oriented with an off-set or alignment angle of an approximately 8° rotation from the user's knee axis “K”, with this off-set or angle also being variable between approximately plus or minus 3.6 degrees (or more depending on the user's physiology), and with approximately 20° to 30° of external rotation along a frontal plane extending along the user's hip and knee.

Further generally illustrated in FIGS. 1, 2 and 7, the prosthetic foot 16 generally will be mounted to the lower or distal/bottom end of the talus. The prosthetic foot 16 may be a customized or custom developed prosthetic foot adapted and/or configured to fit the user, although various conventional design prosthetic foot pads also may be used. The prosthetic foot may also be selected/configured for a “best fit” with the user based on stability and user preferences. For example, the foot may be a solid piece or construction or may include forked or split portions that extend or separate outwardly to provide additional support or balance, as well as other features such as resilient pads, springs or elastomeric members 57 that may be mounted along a lower or heel portion 58 of the prosthetic foot or between the foot and the bottom of the talus.

As further illustrated in FIGS. 1-5A, the dorsi-flexion tendon assist mechanism 14 will be connected between the talus 12 and the tibia section 11, extending therebetween along a rear portion thereof. The dorsi-flexion assist mechanism 14 generally may comprise a passive spring assist, shown in one aspect as including a pneumatic spring assist 60 having a pneumatic or hydraulic cylinder 61 with an extensible rod 62 telescopically received therein. A rotatable connector 63A generally will be provided at a first or proximal end 64 of the cylinder 61, and a similar connector 63B may be provided at the distal end 66 of the cylinder rod 62. As indicated in FIGS. 1, 6A and 6B the connectors 63A and 63B each may include a body 67 that receives a spherical head 68 of a fastener 69. The opposite ends of the fasteners 69 further each may include a threaded portion 71 that may be received within corresponding threaded fastener openings 72 formed in the body of tibia section and the body of the talus, respectively. As will be understood by the skill in the art other, rotatable or moveable connecting mechanisms, such as other types of universal joint type connectors, also may be used. In addition, further will be understood by those skilled in the art that their types of passive spring assist mechanisms, in addition to hydraulic or pneumatic cylinders, also may be used.

The passive spring assist mechanism 14 is configured to provide a preset amount of a spring or biased propulsion force directed in a dorsi-flexion vector 75 (FIGS. 1-2) to assist in push-off or a pre-swing movement such as when the user stands and/or pushes off at the start of locomotion. By way of example, preliminary testing has utilized a passive spring assist force of approximately 25-30 pounds of force. The spring assist force provided by the passive pneumatic spring system mechanism can, however, be varied or adjusted as needed to substantially match or more closely approximate a normal push-off or pre-swing force needed/applied by the user when initiating locomotion or standing and thus more closely approximate or match the user's natural pre-amputation movement/locomotion.

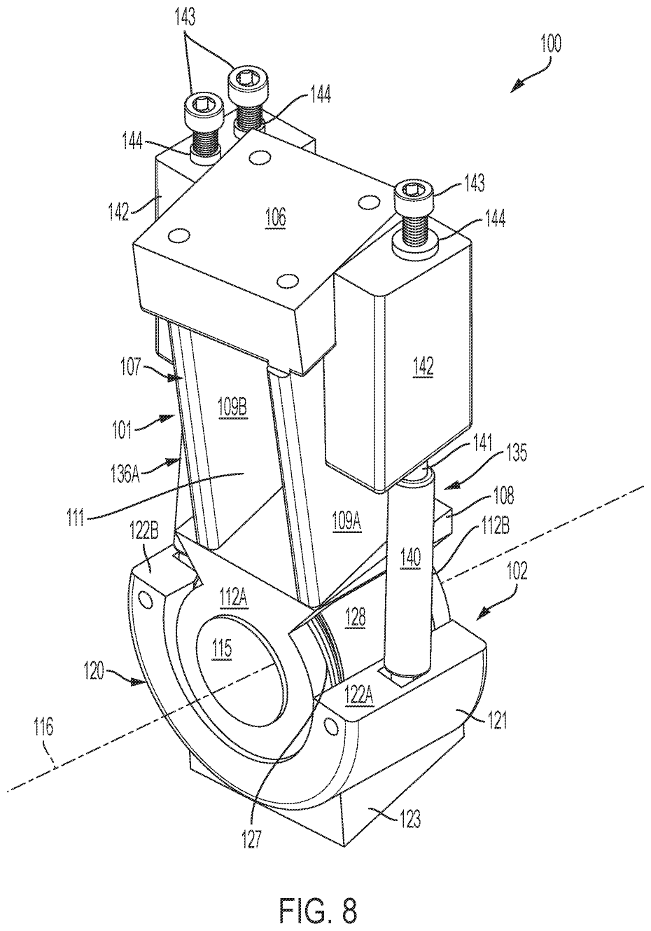

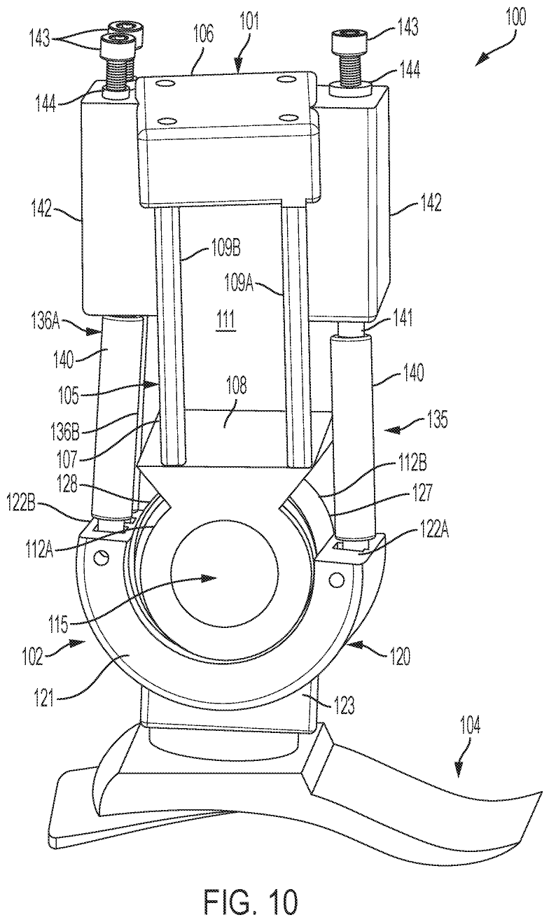

An additional embodiment of the prosthetic ankle 100 is further illustrated in FIGS. 8-10. As indicated, the prosthetic ankle 100 generally will include a tibia section 101 and a talus or ankle joint 102 with a pivoting/moveable connection coupling the ankle joint or talus 102 and the tibia section 101 so as to enable pivoting or other movement between the talus and the tibia section during movement by the user. The prosthetic ankle further may include a prosthetic foot 104 (FIG. 10), such as is discussed above with respect to the prosthetic ankle of FIGS. 1-7 above, that may be moveably mounted to the talus 102 (FIG. 8). The tibia section 101 and talus 102 may incorporate similar constructions and features as the talus and tibia sections shown and discussed above with respect to FIGS. 1-7.

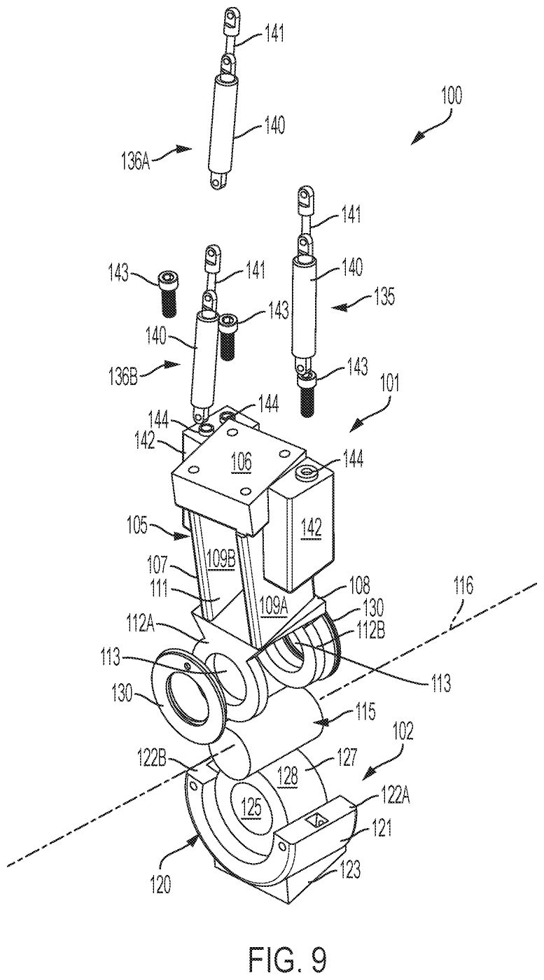

As further indicated in FIGS. 8-10, the tibia section 101 generally may include a body 105 having an open or substantially skeletonized construction, such as for reducing weight. The boy 105 of the tibia section 101 may have an upper or proximal end portion 106, and intermediate body section 107, and a lower or distal end portion 108. As indicated in FIGS. 9 and 10, the intermediate body section 107 of the tibia body may be formed with a substantially open or skeletonized construction, including front and rear-facing portions or plates 109A/109B that are spaced apart so as to define a passage or opening 111 therebetween. The upper or proximal end 106 of the body 105 of the tibia section 101 further may be configured to receive a connector or coupling device, such as a pyramid adapter, or other, similar connector for coupling the prosthetic ankle to a sleeve or corresponding connector such as for attachment of the prosthetic ankle to the user's residual limb.

The lower or distal end portion 108 of the body 105 of the tibia section 101 further may be formed as a clevis or with a substantially C or U shape construction, such as discussed above with respect to the embodiment of FIGS. 1-7, and may include downwardly projecting side portions or legs 112A/112B. The legs 112A/112B generally will be spaced apart so as to define an open area or recess 113 within which the talus 102 of the prosthetic ankle 100 is received as indicated in FIG. 9. The legs of the distal end portion 108 of the tibia section further may only include aligned openings defining a passage therethrough for receipt of a connector rod or pin 115 therethrough, and which defines an ankle joint pivot axis 116 about which the talus 102 pivots with respect to tibia section 101.

As further illustrated in FIGS. 8-10, in one embodiment, the talus 102 may include a body 120 including a base 121, indicated in the FIGS. 8-10 as including a substantially semi-circular or arcuate configuration with front and rear terminal portions 122A and 122B. The lower end 123 of the talus body further may be substantially flat and/or may be configured to engage and attach to a connector by which a prosthetic foot 104 (FIG. 10) may be attached to the talus, such as discussed above with respect to FIGS. 1-7. The talus body 120 (FIG. 9) further may include a substantially circular mid-section 127 having an upper portion that is adapted to be received in the recess defined between the legs 112A/112B of the tibia section, and which generally includes a curved or arcuate upper surface 128 configured to substantially facilitate rotation of the talus or ankle joint with respect to the tibia section. The talus 102 further may be oriented at a slight angle or cant with respect to the tibia section. In addition, the body of the talus 102 further may be angled or arranged at an offset or slanted configuration with respect to its base or lower end portion to match the user's sound limb.

As indicated in FIG. 8, the connector 115 generally may include a rod or pin that will be received within the passage 113 extending through both of the side portions or legs of the tibia body, as well as through a central opening 125 defined through the mid-section 127 of the talus body 120. The connector generally will serve as a pivot or hinge pin about which the talus is able to pivot with respect to the body of tibia section. The connector 115 may be further locked or engaged in place via bearings or lock rings 130, as indicated in FIG. 9, to prevent the connector from being inadvertently dislodged.

As additionally generally illustrated in FIGS. 8-10, the prosthetic ankle 100 further may include one or more spring assist mechanisms, including a front or plantar-assist spring mechanism 135 arranged along the front portion 109B of the tibia body, and one or more rear or dorsi-flexion spring assist mechanisms 136A/136B mounted along the rear side portion 109B of the tibia body. The dorsi-flexion assist mechanisms 136A/136B and plantar-assist spring mechanism 135 may comprise hydraulic or pneumatic cylinders or other types of passive spring assist mechanisms, and may be adjustable so as to provide a desired or variable amount of biased propulsion or resistance force directed along a dorsi-flexion vector and/or a plantar vector, for example to help/assist with normal or natural return swinging movement of the ankle and/or prosthetic foot during locomotion by the user, as well as applying additional stability and assistance for user in a terminal stance or other positions.

As indicated in FIGS. 8-10, in one embodiment, a pair of dorsi-flexion spring assist mechanisms 136A/136B may be used, mounted in a side-by-side arrangement along the rear side portion 109B of the body of the tibia section; and a plantar assist spring mechanism may be provided along the front side portion 109A of the body of the tibia section. It will be understood that fewer or less dorsi-flexion spring assist mechanisms and/or other arrangements or combinations of dorsi-flexion and plantar assist spring assist mechanisms also may be used.

In an embodiment, each of the dorsi-flexion and the plantar assist spring assist mechanisms generally may include a cylinder body 140, with an extensible/retractable cylinder rod 141 that is received within and coupled to a support block 142 on or along an exterior side portion 109A/109B of the tibia body 105. Fasteners 143 such as adjustment screws or other, similar adjustment mechanisms further will be received within openings 144 in the upper portions of the cylinder support blocks 142. The fasteners will engage the ends of the cylinder rods 141 to enable adjustment of the spring assist/resistance force being provided by the dorsi-flexion and/or the plantar assist spring mechanisms. The adjustment mechanisms are adjustments provided by the adjustment screws enable individualized tuning of the systems or resistance force provided by the assist mechanisms to enable variation or adjustment as needed to substantially match more or closely approximate user motion and/or standing during various activities or activity levels as well as to accommodate for differences in movement between different patients/users (i.e., young vs. old patients, more active vs. more sedentary patients).

The foregoing description generally illustrates and describes various embodiments of the present disclosure. It will, however, be understood by those skilled in the art that various changes and modifications may be made to the above-discussed construction of the present disclosure without departing from the spirit and scope of the disclosure as disclosed herein, and that it is intended that all matter contained in the above description or shown in the accompanying drawings shall be interpreted as being illustrative, and not to be taken in a limiting sense.

Furthermore, the scope of the present disclosure shall be construed to cover various modifications, combinations, additions, alterations, etc., above and to the above-described embodiments, which shall be considered to be within the scope of the present disclosure. Accordingly, various features and characteristics of the present disclosure as discussed herein may be selectively interchanged and applied to other illustrated and non-illustrated embodiments of the disclosure, and numerous variations, modifications, and additions further may be made thereto without departing from the spirit and scope of the present disclosure as set forth in the appended claims.

We claim:

1. A prosthetic ankle comprising:
a tibia section;
a talus coupled to the tibia section;
a connector rotatably coupling the talus to the tibia section such that the talus is positioned at an off-set alignment with respect to a transverse plane and with respect to a coronal plane;
a dorsi-flexion tendon linkage coupled to the tibia section and to the talus, the dorsi-flexion tendon linkage positioned to provide a passive assist force; and
a foot connected to the talus, the offset alignment of the talus being selected so as to define a transverse ankle joint axis and a coronal ankle joint axis that substantially match an existing ankle joint alignment of a user when positioned thereon.

2. The prosthetic ankle of claim 1, wherein the connector comprises a pin extending through spaced projections of the tibia section and through the talus so as to enable the talus to be pivoted with respect to the tibia section.

3. The prosthetic ankle of claim 1, wherein the coronal joint axis defined by the talus is offset from the coronal plane by approximately 5-12°.

4. The prosthetic ankle of claim 3, wherein the transverse ankle joint axis defined by the talus is offset from the transverse plane at an angle of approximately 5-12°.

5. The prosthetic ankle of claim 1, wherein a coronal joint axis defined by the talus is aligned at an angle of approximately 8° from a vertical tibia midline of the user, and wherein the transverse ankle joint axis is offset at an angle of approximately 8° from a knee axis of the user.

6. The prosthetic ankle of claim 1, wherein the connector defines a lateral joint axis substantially in vertical alignment with a contralateral biological limb of the user.

7. The prosthetic ankle of claim 6, wherein the lateral joint axis extends through the connector and is in substantially vertical alignment with respect to a tibia midline axis of the user.

8. The prosthetic ankle of claim 1, wherein the dorsi-flexion tendon linkage comprises a pneumatic spring assist mechanism.

9. The prosthetic ankle of claim 1, wherein the dorsi-flexion tendon linkage provides passive assist force of approximately 25-30 lbs. in a dorsi-flexion vector.

10. The prosthetic ankle of claim 1, wherein the dorsi-flexion tendon linkage comprises a passive pneumatic cylinder having a cylinder rod that is extensible therefrom and is pivotally coupled to the talus and tibia section so as to enable approximately 20-25° of movement of the talus in a dorsi-flexion vector, and approximately 25-30° movement in a plantar-flexion vector.

11. A prosthetic ankle comprising:
a tibia section;
a talus coupled to the tibia section;
a connector rotatably coupling the talus to the tibia section such that the talus is positioned at an off-set alignment with respect to a transverse plane and with respect to a coronal plane. the connector including a pin extending through spaced projections of the tibia section and through the talus so as to enable the talus to be pivoted with respect to the tibia section;
a dorsi-flexion tendon linkage coupled to the tibia section and to the talus, the dorsi-flexion tendon linkage positioned to provide a passive assist force; and
a foot connected to the talus, the offset alignment of the talus being selected so as to define a transverse ankle joint axis and a coronal ankle joint axis that substantially match an existing ankle joint alignment of a user when positioned thereon, the coronal joint axis defined by the talus being positioned offset from the coronal plane by approximately 5-12°, and the transverse ankle joint axis defined by the talus being positioned offset from the transverse plane at an angle of approximately 5-12°.

12. The prosthetic ankle of claim 11, wherein the connector defines a lateral joint axis substantially in vertical alignment with a contralateral biological limb of the user, and wherein the lateral joint axis extends through the connector and is in substantially vertical alignment with respect to a tibia midline axis of the user.

13. The prosthetic ankle of claim 11, wherein the dorsi-flexion tendon linkage comprises a pneumatic spring assist mechanism, and wherein the dorsi-flexion tendon linkage provides passive assist force of approximately 25-30 lbs. in a dorsi-flexion vector.

14. The prosthetic ankle of claim 11, wherein the dorsi-flexion tendon linkage comprises a passive pneumatic cylinder having a cylinder rod that is extensible therefrom and is pivotally coupled to the talus and tibia section so as to enable approximately 20-25° of movement of the talus in a dorsi-flexion vector, and approximately 25-30° movement in a plantar-flexion vector.

15. A prosthetic ankle comprising:
a tibia section;
a talus connected to the tibia section;
a connector rotatably connected to the talus to the tibia section such that the talus is positioned at an off-set alignment with respect to one or more of a transverse plane and a coronal plane;
a dorsi-flexion tendon linkage connected to the tibia section and to the talus, the dorsi-flexion tendon linkage positioned to provide a passive assist force of approximately 25-30 lbs. in a dorsi-flexion vector; and
a foot connected to the talus, the offset alignment of the talus being selected so as to define a transverse ankle joint axis and a coronal ankle joint axis that substantially match an existing ankle joint alignment of a user when positioned thereon, and a coronal joint axis defined by the talus being aligned at an angle of approximately 8° from a vertical tibia midline of the user, and wherein the transverse ankle joint axis is offset at an angle of approximately 8° from a knee axis of the user.
